(19) United States
(12) Patent Application Publication
(10) Pub. No.: US 2004/0116409 A1
(43) Pub. Date: Jun. 17, 2004

(54) Ocular therapy

(76) Inventor: Peter A. Campochiaro, Baltimore, MD (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/702,691

(22) Filed: Nov. 6, 2003

Related U.S. Application Data
(60) Provisional application No. 60/425,230, filed on Nov. 8, 2002.

(51) Int. Cl. A61K 31/551

(52) U.S. Cl. 514/219

(57) ABSTRACT

Methods for the treatment of a subject suffering from ERM formation or retinal detachment due to ERM formation comprising administering to a subject suffering from ERM formation or retinal detachment due to ERM formation an effective amount of a staurosporine derivative to treat ERM formation or retinal detachment in the eye of the subject, or a salt thereof, are provided.

Epiretinal membrane (ERM) formation is a proliferation of cells in the retina that causes the production of sheets of cells and extracellular matrix that exert traction on the retina. ERM formation is a common cause of visual impairment. Mild ERMs cause wrinkling and distortion of the retina, and when the macula is involved, this results in metamorphopsia and mild to moderate decreased vision. Severe ERMs result in retinal detachment and severe visual loss, and unless corrected by vitreous surgery can cause blindness.

Increased expression of vascular endothelial growth factor (VEGF) is both necessary and sufficient for the development of retinal neovascularization. The new blood vessels lay down extracellular matrix and recruit glial cells and retinal pigmented epithelial (RPE) cells, resulting in ERMs that can obscure the retina and/or detach it. PDGF is a potent chemoattractant for retinal glia and RPE cells. PDGF B-chain (PDGF-B) is produced by endothelial cells, and endothelial-cell derived PDGF-B is necessary for pericyte recruitment during vascular development. Transgenic mice in which the rhodopsin promoter drives expression of PDGF-B in photoreceptors (rho/PDGF-B mice) develop ERMs consisting of glial cells, endothelial cells and pericytes that cause traction retinal detachment within 2-3 weeks of the onset of transgene expression. The cellular components are similar to those in diabetic membranes and therefore, rho/PDGF-B mice provide a useful model of diabetic traction retinal detachment.

PDGF A-chain (PDGF-A) is produced by retinal ganglion cells and vascular cells and during development PDGF-A stimulates migration of astrocytes into the retina from the optic nerve. The expression of PDGFs in the retina is reduced in adults, but retinal detachment results in increased production of PDGFs by RPE cells, and several lines of evidence have implicated PDGF-A in proliferative vitreoretinopathy (PVR), a disease process in which ERMs and traction retinal detachment occur after retinal reattachment surgery. Transgenic mice in which the rhodopsin promoter drives expression of PDGF-A in photoreceptors (rho/PDGF-A mice) develop ERMs consisting solely of glial cells. Homozygous rho/PDGF-A mice develop slowly progressive retinal detachment, and after detachment there is proliferation of RPE cells resulting in subretinal membranes, and eventually a funnel-shaped detachment. This model mimics many aspects of PVR.

### DETAILED DESCRIPTION OF THE INVENTION

It has now been unexpectedly discovered that receptor kinase inhibitors can treat traction retinal detachment due to ERM formation.

There is provided a method for the treatment of a subject suffering from ERM formation or retinal detachment due to ERM formation comprising administering to a subject suffering from ERM formation or retinal detachment due to ERM formation an effective amount of a staurosporine derivative to treat ERM formation or retinal detachment in the eye of the subject. The methods of the present invention use a composition containing a compound disclosed in U.S. Pat. No. 5,093,330.

The invention also includes methods for the treatment of a subject suffering from ERM formation or retinal detachment due to ERM formation comprising administering to a subject suffering from ERM formation or retinal detachment due to ERM formation an effective amount of a staurosporine derivative in combination with another compound or agent that is not a staurosporine derivative that inhibits the activity of VEGF to treat ERM formation or retinal detachment in the eye of the subject. Such other compounds and agents are known to those of skill in the art, and include, e.g., the compound 1-(4-chloroanilino)-4-(4-pyridylmethyl)phthalazine and its salts, e.g., hydrochloride salts, and other compounds disclosed in U.S. Pat. No. 6,271,233, hereby incorporated by reference herein in its entirety.

Pharmaceutical compositions useful in the practice of the present invention contain staurosporine derivatives such as those disclosed in U.S. Pat. Nos. 5,093,330 and 6,214,819 as the active ingredient, and in the practice of the invention can be administered enterally, nasally, buccally, rectally, topically, orally and parenterally, e.g., intravenous, intramuscular, intravitreal, subconjunctival or subcutaneous administration, to treat ERM and traction retinal detachment due to ERM formation in mammalian subjects, especially humans. The compositions may contain the active ingredient alone or, preferably, the active ingredient along with a pharmaceutically acceptable carrier. The effective dosage of the active ingredient depends on the type of targeted disease, as well as the species, age, weight and physical condition of the subject, pharmacokinetic data, and the mode of administration. Examples of effective oral daily doses in humans are, e.g., between about 1 mg and about 250 mg, between about 10 mg and about 150 mg, between about 12.5 mg and about 150 mg, between about 25 mg and about 150 mg, between about 50 mg and about 150 mg, and between about 100 mg and about 150 mg. Dosages can be repeated on a daily basis as necessary to achieve the desired decrease in ERM formation and retinal detachment. Daily doses can be administered at one time point, or can be divided with the total daily dose being administered over several time points during the day.

When administered topically or intra-vitreally, effective dosage are between about 1 mg and about 250 mg, e.g., between about 10 mg and about 150 mg, between about 25 mg and 50 mg, or about 25 mg per administration.

Ointment type preparations of the staurosporine derivative for topical administration can be prepared containing, e.g., wool fat, white petrolatum, liquid paraffin, polyethylene glycol, e.g., PEG 400, and one or more ophthalmically acceptable preservatives.

Aqueous topical ocular compositions used in the practice of the methods of the invention can be administered, e.g., one, two, three or four times daily, where each administration comprises one, two, three, four or five drops per eye.

The compounds useful in the methods of the invention are administered in an amount effective to decrease in ERM formation and retinal detachment in the retina of a subject suffering from these conditions. Suitable pharmaceutical compositions may have from about 1% to about 95% of the active ingredient. Suitable unit dose forms include coated and uncoated tablets, ampoules, vials, suppositories or capsules. Other suitable dosage forms include injectables, intraocular devices, intravitreal devices, ointments, creams, pastes, foams, tinctures, eye-drops, oral drops, sprays, dispersions and the like. The pharmaceutical compositions useful in the methods of the present invention are prepared in a manner known in the art, for example, by means of conventional mixing, granulating, coating, dissolving or lyophilizing processes.

Solutions of the active ingredient, and also suspensions or dispersions, especially isotonic aqueous solutions, dispersions or suspensions are also useful in the practice of the invention. Suitable useful pharmaceutical compositions containing the active ingredient may have carriers, e.g., mannitol and starch, preservatives, stabilizers, wetting agents, emulsifiers, solubilizers, salts for regulating osmotic pressure, buffers and the like. The compositions are prepared in a manner known in the art, for example, by means of conventional dissolving and lyophilizing processes. A solution or suspension form of the composition may contain viscosity-increasing agents, e.g., sodium carboxymethylcellulose, carboxymethylcellulose, dextran, polyvinylpyrrolidone and gelatins; and solubilizers, e.g., Tween 80 (polyoxyethylene(20)sorbitan mono-oleate; trademark of ICI Americas, Inc, USA).

Suitable carriers include fillers, e.g., sugars, for example, lactose, saccharose, mannitol or sorbitol; cellulose preparations; calcium phosphates, e.g., tricalcium phosphate and calcium hydrogen phosphate; binders, e.g., starches, methylcellulose, hydroxypropyl methylcellulose, sodium carboxymethylcellulose and polyvinylpyrrolidone; and, if desired, disintegrators, e.g., starches, crosslinked polyvinylpyrrolidone, alginic acid or salts thereof. Additional suitable excipients are flow conditioners and lubricants, e.g., silicic acid, talc, stearic acid and salts thereof, such as magnesium or calcium stearate, polyethylene glycol, and derivatives thereof.

In addition to the active ingredients, pharmaceutical compositions useful in the practice of the presently claimed methods may contain suitable pharmaceutically-acceptable carriers comprising excipients and auxiliaries which facilitate processing of the active compounds into preparations which can be used pharmaceutically. Further details on techniques for formulabon and administration may be found in the latest edition of Remington's Pharmaceutical Sciences (Maack Publishing Co., Easton, Pa.).

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato or other plants; cellulose, such as methyl cellulose, hydroxypropylmethyl-cellulose or sodium carboxymethylcellulose; gums including arabic and tragacanth; and proteins, such as gelatin and collagen. If desired, disintegrating or solubilizing agents may be added, such as the cross-linked polyvinyl pyrrolidone, agar, alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol (PEG) and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid or liquid PEG with or without stabilizers.

The present invention also provides for administration of a pharmaceutical composition comprising a solution or dispersion of a staurosporine active ingredient in a saturated polyalkylene glycol glyceride.

The kinase inhibitor active ingredients may be, e.g., any of the staurosporine derivatives described in U.S. Pat. No. 5,093,330. Preferred compounds are N-acylstaurosporines including N-benzoyl staurosporine, N-(3-nitrobenzoyl)staurosporine, N-(3-fluorobenzoyl)staurosporine, N-trifluoracetylstaurosporine, N-phenylcarbamoylstaurosporine, N-(3-carboxypropionyl)staurosporine, N-methylaminothiocarbonylstaurosporine, N-tert-butoxycarbonylstaurosporine, N-(4-carboxybenzoyl)staurosporine, N-(3,5-dinitrobenzoyl)staurosporine, N-(2-aminoacetyl)staurosporine, N-alanylstaurosporine and their pharmaceutically acceptable salts. An especially preferred active ingredient is N-benzoyl staurosporine.

The saturated polyalkylene glycol glyceride may be, for example, a mixture of glyceryl and PEG esters of one or more long chain saturated fatty acids, usually C₈-C₁₈ saturated fatty acids. The acid component of such esters may be, for example, caprylic acid, capric acid, lauric acid, myristic acid, palmitic acid, stearic acid or a mixture of two or more thereof. The PEG component of such esters generally has a molecular weight of 200-2000, preferably 1000-1800, especially 1400-1600. The glycerides, i.e., the glycol-modified glycerides, are usually mixtures of mono, di- and triglycerides and PEG mono- and di-esters.

Preferred polyalkylene glycol glycerides are those having a high hydrophilic-lipophilic balance (HLB) value. Further preferred are glycerides which are mixtures of esters of one or more C₈-C₁₈ saturated fatty acids with glycerol and a PEG having a molecular weight of 1000-2000, preferably 1200-1800, especially 1400-1600. An especially preferred material is available commercially from Galtefosse as Gelucire 44/14; this is a mixture of esters of C₈-C₁₈ saturated fatty acids with glycerol and a PEG having a molecular weight of about 1500, the specifications for the composition of the fatty acid component being, by weight, 4-10% caprylic acid, 3-9% capric acid, 40-50% lauric acid, 14-24% myristic acid, 4-14% palmitic acid and 5-15% stearic acid.

The saturated polyalkylene glycol glycerides are either commercially available or may be prepared by known procedures. For example, they may be obtained by partial alcoholysis of hydrogenated vegetable oils using the polyalkylene glycol or by esterification of the saturated fatty acid, or mixture of such acids, using glycerol and the polyalkylene glycol.

In compositions of the invention, the kinase inhibitor active ingredient is generally present in an amount from 1-30%, preferably 5-25%, especially 10-20%, by weight of the composition.

The compositions of the invention may also contain carriers or adjuncts such as those described in U.S. Pat. No. 5,093,330 or other conventional excipients. For oral administration, the composition may be contained in capsules, usually hard capsules of gelatin or soft capsules of gelatin mixed with a plasticizer, such as glycerol or sorbitol, or may be used as a dispersion in an aqueous medium, such as water, saline solution or a mixture of water with another, water-miscible, pharmaceutically acceptable solvent, for example, in an amount of 0.5-70, preferably 5-50% by weight, optionally together with a preservative, for example, a conventional preservative such as a benzoate, particularly an ester of p-hydroxybenzoic acid, such as the methyl, ethyl, n-propyl, n-butyl or benzyl ester thereof or the sodium salt of the ester and other excipients, such as dispersing agents and suspending agents.

The present invention also provides a method of preparing a pharmaceutical composition as hereinbefore described which comprises melting a saturated polyalkylene glycol glyceride, mixing a kinase inhibitor active ingredient with the molten glyceride and allowing the resulting mixture to solidify.

The glyceride is conveniently melted by heating to a temperature 10-20° C. above its melting point before addition of the kinase inhibitor active ingredient as a powder. Optional excipients may be added to the molten mixture.

When a composition of the invention is to be administered in capsules, for example, orally, the liquid mixture of the kinase inhibitor active ingredient and glyceride may be poured into hard capsules or injected into soft capsules and allowed to solidify therein. Alternatively, the solid solution or solid dispersion obtained on cooling the liquid mixture of the kinase inhibitor active ingredient and glyceride may be re-melted for introduction into capsules. The capsules may contain, for example, from 1-250 mg of the kinase inhibitor active ingredient.

When a composition of the invention is to be administered as a dispersion in an aqueous medium, e.g., water, a saline solution or mixture of water with a water-miscible pharmaceutically acceptable solvent, the solid solution or solid dispersion obtained on cooling the liquid mixture is conveniently broken up and dispersed in the aqueous medium by stirring or by ultrasonication.

Other suitable formulations for the administration of kinase inhibitors according to the methods of the present invention are set out in international patent application publication number WO 00/48571.

Pharmaceutical formulations suitable for parenteral administration may be formulated in aqueous solutions, preferably in physiologically compatible buffers such as Hanks' solution, Ringer's solution or physiologically buffered saline. Aqueous injection suspensions may contain substances which increase the viscosity of the suspension, such as sodium carboxymethyl cellulose, sorbitol or dextran. Additionally, suspensions of the active compounds may be prepared as appropriate oily injection suspensions. Suitable lipophilic solvents or vehicles include fatty oils such as, sesame oil; synthetic fatty acid esters, such as ethyl oleate or triglycerides; or liposomes. Non-lipid polycationic amino polymers may also be used for delivery. Optionally, the suspension may also contain suitable stabilizers or agents which increase the solubility of the compounds to allow for the preparation of highly-concentrated solutions.

The pharmaceutical composition may be provided as a salt and can be formed with many acids including, but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine,0.1-2% sucrose and 2-7% mannitol, at a pH range of 4.5-5.5, that is combined with buffer prior to use.

After pharmaceutical compositions have been prepared, they can be placed in an appropriate container and labeled for treatment of an indicated condition. For administration of the kinase inhibitors disclosed herein, such labeling would include amount, frequency and method of administration.

Pharmaceutcal compositions suitable for use in the invention include compositions where the active ingredients are contained in an effective amount to achieve the intended purpose. The determination of an effective dose is well within the capability of those skilled in the art.

Various biodegradable and biocompatible polymeric matrices comprising the kinase inhibitors set out above, including microcapsules, nanospheres and implants, are useful in the practice of the present invention.

Microspheres are fine spherical particles containing active drugs. They are differentiated from nanospheres primarily by the size of the particle; microspheres have a diameter of less than approximately 1000 μm, while nanospheres are submicronic (<1 μm). Microsphere systems contain either homogeneous monolithic microspheres, in which the drug is dissolved or dispersed homogeneously throughout the polymer matrix, or reservoir-type microspheres, in which the drug is surrounded by the polymer matrix membrane shell. Monolithic and reservoir systems can also be combined. For instance, active drug can be dispersed within, or adsorbed onto, the polymer surface in a reservoir-type microsphere.

Biodegradable polymers can consist of either natural or synthetic materials that vary in purity. Natural polymers include polypeptides and proteins, e.g., albumin, fibrinogen, gelatin and collagen; polysaccharides, e.g., hyaluronic acid, starch and chitosan; and virus envelopes and living cells, e.g., erythrocytes, fibroblasts and myoblasts. Natural materials require cross-linking in the microencapsulation process, leading to the denaturation of the polymer and the embedded drug. As a result, synthetic polymers are most commonly used. Frequently used synthetic polymers include poly(hydroxy) acids, such as polylactic acid, polyhydroxybutryic acid and copoly(lactic/glycolic) acid. These compounds are biocompatible, lack immunogenicity and have physical properties that permit them to be easily shaped (to control the bioerosion rate).

Useful polymers include thermogels, i.e., hydrogels that alter their viscosity in response to changes in temperature. Such thermogels are known in the art and can contain, inter alia, an entangled network of two randomly grafted polymers, e.g., a network of poly(acrylic acid) and a triblock co-polymer containing poly(propylene oxide) (“PPO”) and poly(ethylene oxide) (“PEO”) segments in the sequence PEO-PPO-PEO. This family of polymers goes by the trade name Pluronic polyols. Another Pluronic-based thermogel comprises Pluronic side chains grafted onto a bioadhesive backbone of either poly(acrylic acid) or chitosan. The thermogels useful in the invention are those that are liquid at room temperature, but that form gels at the normal temperature of the human body, i.e., about 37° C.

Colloidal particulate carriers can also be used in the methods of the present invention for delivering kinase inhibitor drugs. Liposomes are the preferred colloidal vehicle, and are composed of a phospholipid bilayer that may act as a carrier for both hydrophilic and hydrophobic medications. Liposomes can be made from, e.g., neutral lipids, charged phospholipids and cholesterol. The addition of an amphophilic polymer, such as PEG onto the surface of a liposome can slow the clearance of liposomes.

The disclosure of all patents, publications, (including published patent applications), and database accession numbers and depository accession numbers referenced in this specification are specifically incorporated herein by reference in their entirety to the same extent as if each such individual patent, publication and database accession number, and depository accession number are specifically and individually indicated to be incorporated by reference.

The present invention is further illustrated with the following examples. However, the example is not to be construed as limiting the invention thereto.

In view of the close relationship between the novel compounds in free form and those in the form of their salts, including those salts that can be used as intermediates, for example, in the purification or identification of the novel compounds, any reference to the free compounds hereinbefore and hereinafter is to be understood as referring also to the corresponding salts, as appropriate and expedient.

### EXAMPLES

Mice are treated humanely in accordance with Association for Research in Vision and Ophthalmology guidelines for the use of animals in ophthalmic and vision research. In the Examples, the term “P(x)”, where X is a number, means postnatal day, e.g., P7 means 7 days after birth. Methods for the generation and characterization of rho/PDGF-A and rho/PDGF-B transgenic mice are known to those of ordinary skill. Hemizygous rho/PDGF-B, line 1 mice in a C57BU6 background and homozygous rho/PDGF-A, line 2 mice in a C57BU6 background are each divided into 5 groups and starting on P7 are treated by gavage once a day with vehicle or vehicle containing 50 mg/kg of PKC412, PTK787, SU1498 or Imatinib mesylate. Rho/PDGF-B mice are euthanized at P12 to assess ERM formation by staining with Griffonia simplicifolia lectin (GSA), which selectively stains vascular cells, or at P21 to assess for retinal detachment. Homozygous rho/PDGF-A mice are euthanized at P40 to assess ERM formation by immunohistochemical staining for glial fibrillary acidic protein (GFAP) or at P50 to assess for retinal detachment.

PKC412 is an inhibitor of the kinase activity of several isoforms of PKC, VEGF receptors, PDGF receptors and ckit, but not receptors of several other growth factors that have been tested. PTK787 is an inhibitor of the kinase activity of VEGF receptors, PDGF receptors, and ckit, but not receptors of several other growth factors that have been tested. SU1498 is an inhibitor or the kinase activity of VEGF receptors. Imatinib mesylate is an inhibitor of the kinase activity of PDGF receptor kinase, ckit and vAbl, but not the receptors of several other growth factors.

Rho/PDGF-B mice are euthanized and eyes are rapidly removed and frozen in optimal cutting temperature embedding media (OCT; Miles Diagnostics, Elkhart, Ind.). Ten μm frozen sections are fixed with 4% paraformaldehyde for 30 minutes, washed with 0.05M Tris-buffered saline (TBS), pH 7.6. Slides are incubated in methanol/H₂O₂ for 10 minutes at 4° C, washed with 0.05 M TBS, and incubated for 30 minutes in 10% normal porcine serum. Slides are incubated 2 hours at room temperature with biotinylated GSA (Vector Laboratories, Burlingame, Calif.) and after rinsing with 0.05 M TBS, they are incubated with avidin coupled to peroxidase (Vector Laboratories) for 45 minutes at room temperature. After a 10-minute wash in 0.05 M TBS, slides are incubated with diaminobenzidine (Research Genetics) to give a brown reaction product and counterstained with hematoxylin and eosin.

Immunohistochemical staining of retinas for GFAP labels astrocytes and activated Muller cells. Homozygous rho/PDGF-A mice are euthanized and eye are frozen in OCT. Ten μm frozen sections are fixed with 4% paraformaldehyde for 30 minutes, washed with 0.05 M TBS, incubated in methanol/H₂O₂ for 10 minutes at 4° C., and washed with 0.05 M TBS. Specimens are blocked with 10% normal goat serum (NGS) in 0.05 M TBS for 30 minutes at room temperature and then incubated with 1:500 rabbit anti-bovine GFAP in 1% NGS/0.05 M TBS and incubated in biotinylated goat anti-rabbit antibody for 30 minutes. After washing, the slides are incubated in streptavidin-phosphatase and developed with HistoMark Red (Kirkegaard and Perry, Gaithersburg, Md.) according to the manufacturer's instructions. Sections are dehydrated and mounted with Cytoseal.

To perform quantitative assessments, 10 μm serial sections are cut through an entire eye starting with sections that included the iris root on one side of the eye and proceeding to the iris root on the other side. Every tenth section, roughly 100 μm apart, is stained with GSA (rho/PDGF-B mice) or anti-GFAP (homozygous rho/PDGF-A mice). Sections are examined with an Axioskop microscope with the examiner masked with respect to treatment group. For assessment of the amount of ERM, images are digitized using a 3 CCD color video camera and a frame grabber. Image-Pro Plus software is used to delineate GSA- or GFAP-stained cells in the retina and their area is measured. The mean of the measurements from each eye is used as a single experimental value. For assessment for retinal detachment, sections are examined and graded as to the presence of partial or total retinal detachment. If all stained sections showed total retinal detachment, then the eye is graded as total retinal detachment. If any of the sections show at least a partial retinal detachment, but all sections did not show total retinal detachment, then the eye is graded as partial retinal detachment. If none of the sections show any retinal detachment, then the eye is graded as no retinal detachment.

For analysis of area measurements, data are analyzed using either a generalized linear model with generalized estimating equations (GEE) or analysis of variance (ANOVA) model. The generalized linear model with GEE is analogous to the ANOVA model; the primary difference is that the allows for correlated data from right and left eyes to be included from direct modeling of the correlation, while the ANOVA model requires the data to be independent, hence right and left eye values must be averaged prior to analysis. Findings are similar with each type of analysis and therefore the generalized linear model with GEE is reported. To adjust for multiple comparisons, a p-value of 0.0125 is required for statistical significance.

For comparisons between vehicle-treated eyes and eyes in the other treatment groups with regard to retinal detachments, data are analyzed using logistic regression with GEE to account for correlation between eyes. To adjust for multiple comparisons, a p-value of 0.0125 is required for statistical significance.

### Example 1

PKC412 and PTK787 Reduce ERM Formation in rho/PDGF-B Mice

Starting at P7, hemizygous rho/PDGF-B mice are given vehicle or vehicle containing 50 mg/kg of PKC412, PTK787, SU1498 or Imatinib mesylate once a day by gavage. Untreated rho/PDGF-B mice consistently develop prominent ERMs by P12 that are best illustrated by staining retinal sections with GSA, and consistent with those previous results extensive GSA-stained ERMs are seen in the eyes of mice treated with vehicle. In contrast, eyes from mice treated with PKC412 or PTK787 have little ERM formation. Eyes from mice treated with SU1498 or Imatinib mesylate have extensive ERM formation, similar to that seen in vehicle-treated eyes. Measurement of the area of GSA staining by image analysis showed significantly smaller areas in PKC412- and PTK787-treated mice compared to vehicle-treated mice, whereas mice treated with SU1498 or Imatinib mesylate showed no significant difference.

### Example 2

PTK787 and PKC412 Reduce Retinal Detachments in rho/PDGF-B Mice

At P21, vehicle-treated mice have extensive ERM formation with multiple layers of ectopic cells in the inner retina and more than 80% of eyes have folding of the outer retina and retinal detachment, with total funnel-shaped detachments in about ⅓ of eyes. Mice treated with 50 mg/kg of PKC412 or PTK787 by gavage between P7 and P21 have mild ERM formation and roughly 50% of the eyes have a normal appearing outer retina. Only 10% of eyes from mice treated with PKC412, and 15% from mice treated with PTK787 have total retinal detachments, compared to 35% of eyes in mice treated with vehicle. Mice treated with SU1498 or Imatinib mesylate have extensive ERM formation and nearly 90% have at least partial retinal detachment. Sixty percent of eyes from mice treated with SU1498 and 30% from mice treated with Imatinib mesylate have total retinal detachment.

Due to the many experimental groups requiring multiple comparisons, the decrease in total retinal detachments in the PKC412 and PTK787 groups is not considered statistically significant. An independent experiment is performed to compare treatment with PKC412 to treatment with vehicle. Roughly 10% of eyes of mice treated with PKC412 compared to 55% of eyes of mice treated with vehicle have total retinal detachment at P21, a difference that is highly statistically significant.

### Example 3

PKC412 and PTK787 Decrease ERM Formation and Retinal Detachment in Homozygous rho/PDGF-A Mice

Homozygous rho/PDGF-A mice (rho/PDGF-M mice) develop glial ERMs that are slowly progressive and often result in traction retinal detachment between 1 and 2 months of age. At P40, eyes from mice treated with vehicle have a thick layer of GFAP-stained cells on the surface of the retina and within the inner nuclear layer. Eyes from mice treated with PKC412 have a layer of glial cells on the surface of the retina, but none or few in the inner nuclear layer and the total area of GFAP staining per section is significantly less than that in vehicle treated eyes. Eyes from mice treated with PTK787 have a layer of GFAP-positive cells on the surface of the retina and occasional clumps in the inner nuclear layer, but the total area of GFAP staining per section is significantly less than that in vehicle-treated eyes. Retinas from mice treated with SU1498 or Imatinib mesylate showed no significant difference from the retinas of vehicle-treated mice in GFAP staining.

At P50, the majority of eyes from vehicle-treated mice (more than 60%) showed total, funnel-shaped detachments, whereas the majority of eyes from PKC412- or PTK787-treated mice showed no detachment. Only 10-15% of eyes in the PKC412 or PTK787 groups have total retinal detachments, which is significantly less than that seen in the vehicle group. There is no decrease in total retinal detachments in mice treated with SU1498 compared to those treated with vehicle, and although there are fewer severe detachments in mice treated with Imatinib mesylate, the difference did not meet the rigorous criterion required for statistical significance given the need for multiple comparisons among the 5 groups.

What is claimed is:

1. A method for the treatment of a subject suffering from ERM formation or retinal detachment due to ERM formation comprising administering to a subject suffering from ERM formation or retinal detachment due to ERM formation an effective amount of a staurosporine derivative to treat ERM formation or retinal detachment in the eye of the subject, or a salt thereof.

2. The method of claim 1, wherein said subject is a human.

3. A method according to claim 1, wherein said staurosporine derivative is a compound of formula (I), wherein formula (I) is 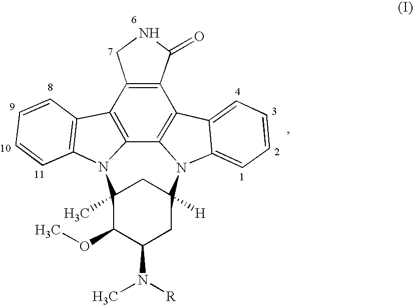
wherein R represents a hydrocarbyl radical RO or an acyl radical Ac, or a salt thereof.

4. The method of claim 3, wherein said hydrocarbyl radical is an acyclic, carbocyclic, carbocyclic-acyclic, heterocyclic or heterocyclic-acyclic hydrocarbyl radical.

5. The method of claim 4, wherein said acyclic hydrocarbyl radical is a radical of a C₁-C₂₀alkyl radical, C₂-C₂₀hydroxyalkyl radical of which the hydroxy group is in any position other than the 1-position, cyano-(C₁-C₂₀)alkyl radical, carboxy-(C₁-C₂₀)alkyl radical of which the carboxy group, or C₃-C₂₀alkenyl radical of which the free valency is not at the same carbon atom as the double bond.

6. The method of claim 4, wherein said carbocyclic hydrocarbyl radical is a radical of mono-, bi- or poly-cyclic cycloalkyl, cycloalkenyl, cycloalkandienyl and aryl.

7. The method of claim 4, wherein said carbocyclic-acyclic radicals is an acyclic radical that carry one or more of carbocyclic radicals, and said heterocyclic radical and heterocyclic-acyclic radical are monocyclic, bicyclic, polycyclic, aza-, thia-, oxa-, thaza-, oxaza-, diaza-, triaza- and tetraza-cyclic radicals of aromatic character.

8. The method of claim 3, wherein said acyl radical is an optionally functionally modified carboxylic acid, organic sulfonic acid or optionally esterified phosphoric acid.

9. The method of claim 3, wherein said acyl radical has the formula
Z-C(═W)—,
wherein
W is oxygen, sulfur or imino; and
Z is hydrogen, C₁-C₇alkyl, amino, phenyl, pyridyl, furyl, thienyl, imidazolyl, quinolyl, isoquinolyl, benzofuranyl or benzimidazolyl.

10. The method of claim 3, wherein said acyl radical has the formula
RbO—CO—,
wherein Rb is hydrogen, benzoyl or a C₁-C₁₉alkyl radical.

11. The method of claim 3, wherein said acyl radical has the formula
RO—O—CO—,
wherein RO is an acyclic, carbocyclic, carbocyclic-acyclic, heterocyclic or heterocyclic-acyclic hydrocarbyl radical.

12. The method of claim 3, wherein said acyl radical has the formula 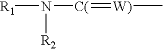
wherein R₁ and R₂ are, independently, selected from hydrogen and unsubstituted acyclic C₁-C₇hydrocarbyl.

13. The method of claim 3, wherein said acyl radical has the formula 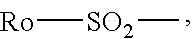
wherein Ro is a hydrocarbyl radical.

14. The method of claim 8, wherein said acyl radical has the formula 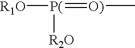
in which R₁ and R₂ are, independently, selected from hydrogen and unsubstituted acyclic C₁-C₇hydrocarbyl.

15. The method of claim 3, wherein said staurosporine derivative is a member selected from the group consisting of N-(3-carboxypropionyl)-staurosporine, N-benzoyl-staurosporine, N-trifluoracetyl-staurosporine, N-methylaminothiocarbonyl-staurosporine, N-phenylcarbamoyl-staurosporine, N-(3-nitrobenzoyl)-staurosporine, N-(3-fluorobenzoyl)-staurosporine, N-tert-butoxycarbonyl-staurosporine, N-(4-carboxybenzoyl)-staurosporine, N-(3,5-dinitrobenzoyl)-staurosporine, N-alanyl-staurosporine, N-ethyl-staurosporine, N-carboxymethyl-staurosporine, N-[(tert-butoxycarbonylamino)-acetyl]-staurosporine, N-(2-aminoacetyl)-staurosporine and salts thereof.

16. The method of claim 1, wherein said staurosporine derivative is N-benzoyl-staurosporine or a salt thereof.

17. The method of claim 3, further comprising the administration of a second compound that inhibits VEGF activity wherein said second compound does not have a structure as set out in formula (I).

18. The method of claim 16, further comprising the administration of a second compound that inhibits VEGF activity wherein said second compound does not have a structure as set out in formula (I).

19. The method of claim 17, wherein said second compound is 1-(4-chloroanilino)-4-(4-pyridylmethyl)phthalazine and its salts.

20. The method of claim 18, wherein said second compound is 1-(4-chloroanilino)-4-(4-pyridylmethyl)phthalazine and its salts.

21. The method of claim 1, wherein said staurosporine derivative is administered topically to the eye.

22. The method of claim 3, wherein said compound is administered topically to the eye.

23. The method of claim 16, wherein said staurosporine derivative is administered topically to the eye.

24. The method of claim 17, wherein said staurosporine derivative and said second compound are administered topically to the eye.

25. The method of claim 18, wherein said staurosporine derivative and said second compound are administered topically to the eye.

26. The method of claim 19, wherein said staurosporine derivative and said second compound are administered topically to the eye.

27. The method of claim 20, wherein said staurosporine derivative and said second compound are administered topically to the eye.
